(19) United States
(12) Patent Application Publication
(10) Pub. No.: US 2008/0009900 A1
(43) Pub. Date: Jan. 10, 2008

(54) SURGICAL GRASPING DEVICE

(75) Inventors: Malcolm Heaven, Dana Point, CA (US); William T. Scott, San Diego, CA (US); Brad F. Giannotti, Coudersport, PA (US); John P. Greelis, Carlsbad, CA (US); Joseph C. Tauro, Brick, NJ (US); Matthew P. France, Morristown, NJ (US); Michael Green, Pleasanton, CA (US); Alfred Martinetti III, Pine Beach, NJ (US)

Correspondence Address: KNOBBE MARTENS OLSON & BEAR LLP, 2040 MAIN STREET, FOURTEENTH FLOOR, IRVINE, CA, 92614, US

(73) Assignee: KFx Medical Corporation, Carlsbad, CA (US)

(21) Appl. No.: 11/760,621

(22) Filed: Jun. 8, 2007

Related U.S. Application Data
(60) Provisional application No. 60/812,836, filed on Jun. 12, 2006.

(51) Int. Cl. A61B 17/28

(52) U.S. Cl. 606/207

(57) ABSTRACT

Disclosed herein are methods and devices for securing soft tissue to a rigid material such as bone. A surgical device having a jaw assembly with a sharpened beak-shaped tip and an aperture suitable for receiving a suture anchor is described. The surgical device includes slots in the side of the aperture through which suture that is attached to the anchor can be disengaged from the device.

### RELATED APPLICATIONS

This application claims the benefit of U.S. Provisional Patent Application No. 60/812,836, filed on Jun. 12, 2006, the disclosure of which is hereby incorporated by reference in its entirety.

### BACKGROUND OF THE INVENTION

1. Field of the Invention

The present invention relates to medical devices and procedures. More particularly, the present invention relates to devices and methods for grasping or gripping soft tissue and passing sutures through the soft tissue in order to secure the soft tissue to a rigid material such as bone.

2. Description of the Related Art

There are several medical procedures where a surgeon needs to attach soft tissue such as tendons or other soft connective tissue to bone. One common example is a torn rotator cuff, where the supraspinatus tendon has separated from the humerus causing pain and loss of ability to elevate and externally rotate the arm. To repair a torn rotator cuff, typically a surgical procedure is used to suture the torn tendon to the bone using a variety of methods. Some procedures utilize large incisions and involve complete detachment of the deltoid muscle from the acromion. Small diameter holes are made in the bone for passing suture material through the bone to secure the tendon. Such large incision procedures are traumatic, causing prolonged pain and recovery time.

Other procedures make small incisions and use arthroscopic techniques to attach sutures using either small diameter holes or a bone anchor. In these types of procedures, a surgical instrument known as a grasper may be used to hold the tissue in place so that it is easier to a suture through the tissue. However, it is difficult to manipulate sutures within the surgical site using arthroscopic techniques. In addition, when knot tying is used to secure the suture to a bone anchor, it is difficult to properly adjust the tension of the suture while tightening the knot. Similarly, when the suture is attached to a bone anchor prior to insertion of the anchor into the bone, it is difficult to judge the appropriate point of attachment so that the suture will be properly tensioned upon insertion of the bone anchor into the bone. In order to alleviate some of the above-described difficulties, methods and devices to allow easy arthroscopic attachment of sutures have been proposed. One example of these methods and devices may be found in U.S. patent application Ser. No. 11/143,007 (published as U.S. Pat. Pub. No. 2006-0004364), the content of which is hereby incorporated by reference in its entirety. Although many of the suture attachment issues have been addressed, one of the problems that remains is how to properly locate suture anchors when inserting them through the soft tissue and into the bone. In addition, often times when inserting suture anchors through soft tissue, the soft tissue has a tendency to move during penetration, resulting in an inaccurate placement of the suture anchor in either the bone or the soft tissue. Thus, there is a need for methods and devices that allow easy arthroscopic insertion of suture anchors in precise locations that allow for the attachment of a suture to a bone anchor after the anchor is inserted into the bone without the use of knot tying.

### SUMMARY OF THE INVENTION

The system, method, and devices of the invention each have several aspects, no single one of which is solely responsible for its desirable attributes. Without limiting the scope of this invention, several of its features will now be discussed briefly.

In an embodiment a surgical device for use in grasping tissue, is provided. The surgical device may include a handle assembly and a jaw assembly. A shaft may connect the handle assembly to the jaw assembly. The jaw assembly may include two jaws. The first jaw may have a first aperture with a first aperture slot configured to allow disengagement of a suture passing through the slot, while the second jaw may also have an aperture. The first jaw may further include a spike extending in the direction of the second jaw when the jaws are juxtaposed. The second jaw may also include an aperture to receive the spike. The underside of the shaft and the underside of the second jaw may include a protrusion. The first jaw may also include a sharpened tip adapted to facilitate percutaneous insertion of the jaw assembly.

In another embodiment, a method of attaching soft tissue to bone is provided. The method includes grasping the soft tissue with jaws of a surgical device. The jaws of the surgical device may include an aperture. The method further may include passing an anchor with a pre-attached suture through the aperture, and inserting the suture anchor into the bone. The method may further include placing a tissue augment over the soft tissue prior to grasping the soft tissue, then grasping both the tissue augment and the soft tissue with the jaws of the surgical device. The method may further include passing the suture over the tissue augment and the soft tissue and attaching the suture to a second suture anchor. Prior to inserting the suture anchor into the bone, a protrusion on the surgical instrument may be placed into an indentation formed in the bone.

In still another embodiment, a tissue grasper and bone anchor are combined and configured to form a structure having an upper jaw with an aperture which contacts a first surface of soft tissue. The tissue grasper may also include a lower jaw which contacts a second surface of the soft tissue from an opposite side of the soft tissue. The lower jaw also may include a second aperture. The structure may also have a bone anchor extending through the first aperture, second aperture, and soft tissue positioned between the upper and lower jaws of the tissue grasper. The upper and lower jaws may also include a slot in a side of the first and second apertures, respectively.

### BRIEF DESCRIPTION OF THE DRAWINGS

FIG. 1 depicts a perspective view of a grasper device with right offset jaws in a clamped position.

FIGS. 2A, 2B, and 2C depict a top view of a grasper device with non-offset, right offset, and left offset jaws, respectively.

FIG. 3 depicts a side view of the grasper device.

FIGS. 4A and 4B depict a front view of a grasper with the elongated shaft extending toward the viewer.

FIG. 5A depicts a perspective view of a grasper device configured with straight jaws and a spike on the upper jaws having a corresponding hole in the lower jaw.

FIG. 5B depicts a perspective view of a grasper device with left offset jaws in a released position.

FIGS. 6A and 6B depict close up views of the jaws from FIGS. 5A and 5B.

FIG. 7 depicts a grasper clamping soft tissue.

FIGS. 8A-8H depict a grasper receiving a suture anchor.

FIG. 9 depicts a bottom view of a suture extending from the suture anchor through an aperture in the jaws of a grasper.

FIG. 10 depicts a top view of a suture extending from the suture anchor through an aperture in the jaws of a grasper.

FIG. 11 depicts a suture being slidably removed from the aperture in a grasper.

FIG. 12 depicts a suture after it is removed from the jaws of a grasper.

### DETAILED DESCRIPTION OF THE CERTAIN EMBODIMENTS

In various embodiments, soft tissue may be attached to bone utilizing one or more bone anchors with suture attached thereto in conjunction with a surgical grasper device that allows the surgeon to puncture the tendon at an angle while holding it at a desired location relative to the bone. As used herein, “suture” refers to any flexible structure that can be stretched between two or more anchors and includes, without limitation, traditional suture material, single or multiple stranded threads, or a mesh structure. A “suture” may also take the form of an acellular, collagen membrane or other biologic tissue augment such as described in U.S. Application Publication No. 2006/0067967, which is incorporated herein by reference in its entirety, which may provide a scaffold or support matrix for cellular ingrowth to allow soft tissue to reconstruct itself Suitable biologic tissue augments that are commercially available include, but are not limited to, those available under the trade names TISSUEMEND® (TEI Biosciences Inc., Boston, Mass.), RESTORE® (Depuy, Warsaw, Ind.), GRAFT JACKET® (Wright Medical, Arlington, Tenn.), and CUFF PATCH™ (Organogenesis Inc., Canton, Mass.). The membrane may be used in conjunction with other types of sutures to provide additional support in areas where the tissue is weakened. The augment may also be used to bridge gaps or span a defect between soft tissue including ligaments and tendons as well as gaps between the ligament or tendon to bone insertion points. In some embodiments, in order to effectively attach soft tissue to bone, the suture is passed through the soft tissue in a specific area. In order to help position to soft tissue and/or tissue augments properly in relation to the suture or bone anchors, a surgical instrument may be used to grasp the soft tissue or tissue augment or both and maneuver it into position.

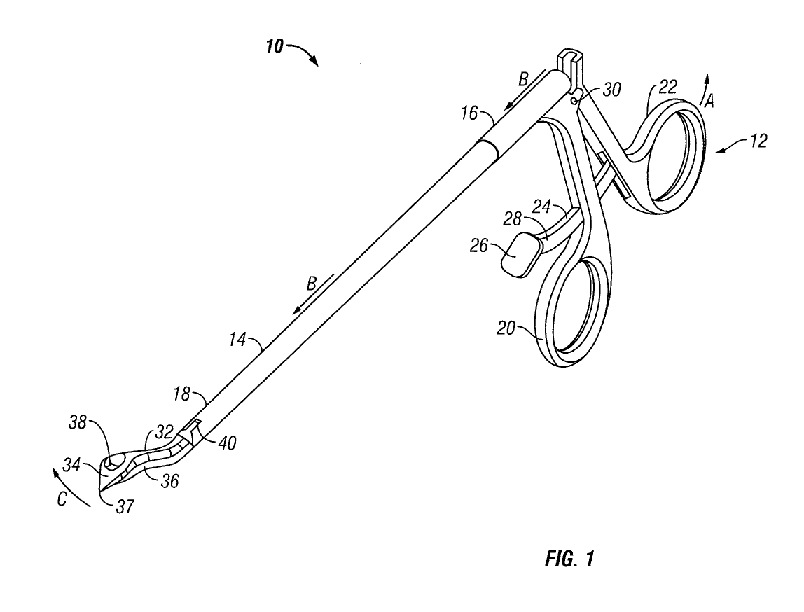

FIGS. 1-6 depict an example of a surgical device 10 which may be used to grasp soft tissue. Surgical device 10 includes a handle assembly 12 and an elongated shaft 14 extending distally from the handle assembly 12. The elongated shaft 14 may include a proximal end 16 located adjacent to the handle assembly 12, and a distal end 18 located at the far end of the elongated shaft 14. The handle assembly 12 may include a thumb loop portion 20 and a finger loop portion 22 which are designed to receive a finger and a thumb of an operator of the surgical device 10 during operation. In some embodiments, the thumb loop 20 may be stationary relative to elongated shaft 14, while the finger loop 22 may be moveable relative to the thumb loop 20. In other embodiments, the finger loop 22 may be stationary, while the thumb loop 20 is moveable relative to the elongated shaft 14. Those of skill in the art will appreciate that a surgeon may manipulate the handle assembly 12 in a variety of ways using a thumb or various fingers in either thumb loop portion 20 or finger loop portion 22. In some embodiments, the surgeon may use two hands to manipulate the handle assembly 12.

The handle assembly 12 may also include a ratcheting or locking device 24 extending out of the inner portion of the finger loop portion 22 through a hole in the body of the thumb loop portion 20 and out beyond the thumb loop portion in toward the distal end of the elongated shaft 14. The ratcheting device 24 may include grooves 28 which may be used to lock the thumb loop portion 20 relative to the finger loop portion 22 by engaging a surface of the hole in the body of the thumb loop portion 20 or other ratchet engaging device. The ratcheting device 24 may be used to lock the thumb loop portion 20 relative to the finger loop portion 22 at any position from being fully open with the thumb loop and finger loops spaced to their maximum, to being fully closed with the thumb loop 20 and finger loop 22 moved very close to each other. Although in the embodiment described above the ratcheting device 24 is engaged by the surface of the hole in the body of the thumb loop portion 20, one of skill in the art will readily appreciate that the handle portion may take various configurations. For example, the ratcheting device 24 may be affixed to the thumb portion, and may engage a surface in hole through the finger loop portion 22. Alternatively, any other suitable structure for locking or retaining the thumb loop 20 and the finger loop 22 relative to each other can be used.

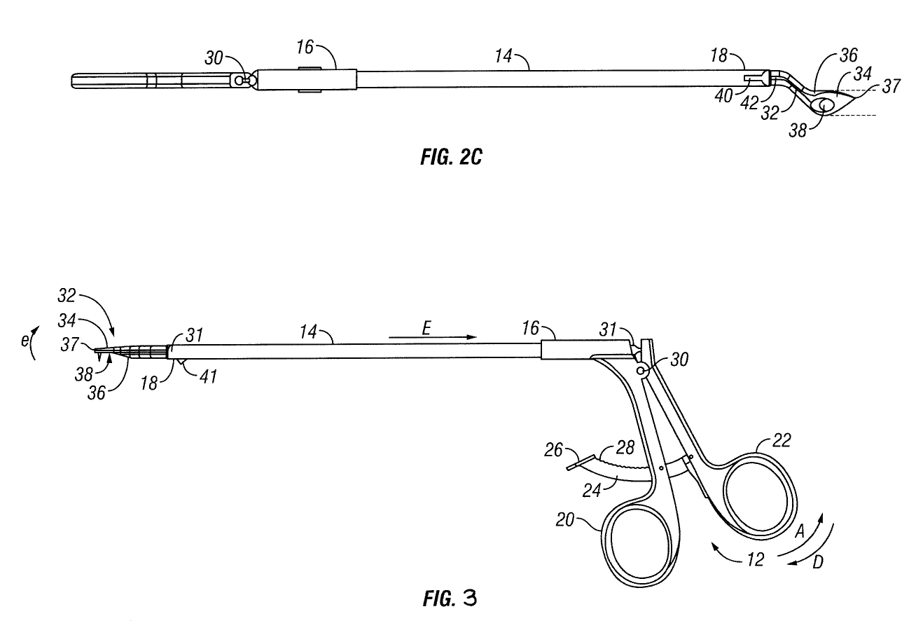

The thumb loop portion 20 and the finger loop portion 22 may be moved relative to one another by virtue of a handle pivot assembly 30, which may be a pin assembly, or some other mechanism that serves as an axial element for movement of the thumb and finger loops. The pivot assembly 30 may also be attached to transmitting rod 31 (shown in FIG. 3) which starts at the proximal area of elongated shaft 14 and extends inside the shaft to a jaw assembly 32 located at the distal end 18 of the shaft.

Although the handle assembly 12 has been described by reference to specific structures, those of skill in the art will appreciate that any of the well-known surgical instrument handle designs may be utilized.

The jaw assembly 32 may include an upper jaw 34 and a lower jaw 36 which are connected to one another through a jaw actuator assembly 40. The lower jaw 36 may be fixedly attached to the elongated shaft 14. Alternatively, the lower jaw 36 may be pivotably attached to the elongated shaft. The upper jaw 34 may also be fixedly attached to the elongated shaft 14, or may alternatively be pivotably attached to the elongated shaft. In either event, the structure provides relative pivotal or clamping motion between the upper jaw 34 and the lower jaw 36. In one embodiment, the distal tip of the upper jaw 34 may be shaped in the form of a sharpened beak 37, which may be sharpened to allow for percutaneous insertion of the jaw assembly. Jaw assembly 32 may also include an aperture 38 in each of upper law 34 and lower jaw 36 positionally aligned to provide a clear path though the body of the jaw assembly 32. In some embodiments, the apertures in the upper and lower jaw may be aligned such that a device passing through them, a suture anchor for example, must pass through at an acute angle to the plan of the jaws. Thus in such an embodiment, the lower jaw aperture is not directly below the upper jaw aperture, but instead is offset slightly. Those of skill in the art will appreciate that the apertures 38 may be aligned in a variety of configurations to provide any desired angle of insertion, including perpendicular, acute, and obtuse angles.

The jaw actuator assembly 40 may be in the form of a small pivot assembly using a small pin to connect the proximal ends of each of the lower jaw 36 and the upper jaw 34 to each other so that they may pivot relative to one another. Alternatively, the pin may pivotably connect either the lower jaw 30 or the upper jaw 34 to the elongated shaft 14 with the other jaw being fixedly connected to the elongated shaft 14. In one embodiment, the transmitting rod 31 is linked to the upper jaw 34 on one end, and to the finger loop portion 22 of handle assembly 12 on the other end. By virtue of its connection to both the jaw assembly and the handle assembly, movement of the handle assembly can be transmitted through transmitting rod 31, or any other suitable force-transmitting structure, to the jaw assembly. Other suitable force-transmitting structures can include, for example, one or more cables, a threaded assembly such as an acme screw, or an electromechanical actuator.

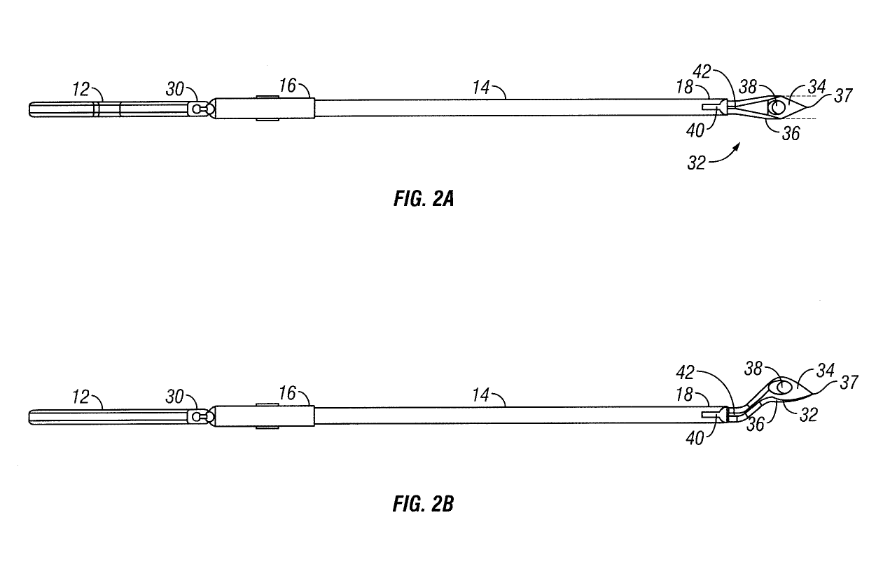

By way of example and not of limitation, the transmitting rod 31 may be coupled to the finger loop portion of the handle assembly 12 such that movement of the finger loop portion in the direction of arrow A causes the transmitting rod 31 to move in the direction of arrow B. The transmitting rod, coupled to the upper jaw portion 36 of the jaw assembly 32, by moving in the direction of arrow B causes the upper jaw portion to open by moving in the direction of arrow C. Conversely, when the finger loop portion of the handle assembly 12 moves back toward the thumb loop portion, the transmitting rod causes the upper jaw 34 to clamp back down on the lower jaw 36. In various embodiments, the surgical device 10 can be configured for use by the left-hand and the right hand respectively. In addition, the jaws of the grasper device 10 may be offset from the elongated shaft 14 or extend straight out from the shaft to provide additional maneuverability and effectiveness in grasping tissue. By way of example and not of limitation, FIG. 2A depicts a top view of a grasper device having straight jaws, while FIGS. 2B and 2C depict views of a grasper device having left and right offset jaws respectively. Offset jaw configurations such as those depicted in FIGS. 2B and 2C allow the surgeon to better clamp tissue of varying shape from a static entry portal or position. Typically, the grasper with right offset jaws is handled using a surgeon's right hand while a grasper device having left offset jaws may be handled by a surgeon's left hand. The straight configuration may be suitably handled by either the right or left hand of the surgeon.

Referring now to FIG. 3, a side view of one exemplary embodiment of a surgical device 10 is provided in which the finger loop portion 22 has been fully moved toward the thumb loop portion 20 in the direction of arrow D, resulting in the transmitting rod 31 to move the direction or arrow E. The movement of the transmitting rod 31 in the direction of arrow E causes the upper jaw 34 of jaw assembly 32 to clamp down against the lower jaw 36 as shown. FIG. 3 also shows an optional underside protrusion 41 which may take the form of a spike that extends downwardly out of the elongated shaft 14 or the jaw assembly 32. The underside protrusion may be used as an anchoring device for the surgical instrument when it is placed against soft tissue or bone as will be described in further detail below. Any suitable shape and number of underside protrusions may be used to stabilize the jaw assembly 32 against bone or tissue. In one embodiment, the underside protrusion 41 may be set into a tapped hole formed in the bone. The tap may be placed into the bone by a needle such as a spinal needle which may be placed against the bone and tapped to create a small indentation in the bone. The lower jaw protrusion 41 may then fit into the tap, which may help keep the surgical device 10 relatively immobile.

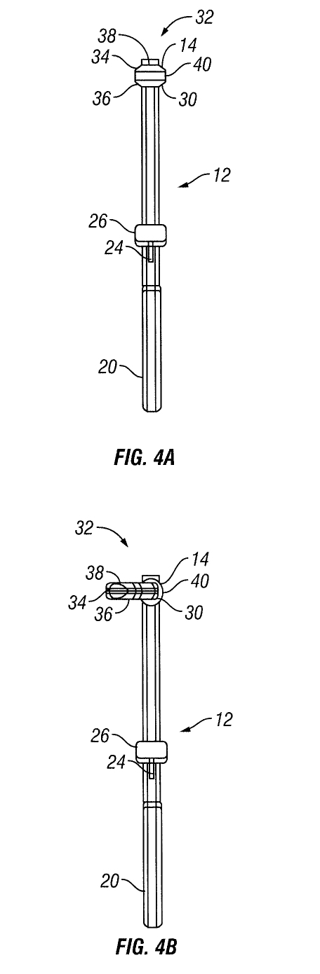

Referring now to FIGS. 4A and 4B, a front view of a surgical device 10 is provided with the elongated shaft extending toward the viewer. The surgical device 10 shown in FIG. 4A is a straight jaw configuration, while the device depicted in FIG. 4B is an offset configuration, in which the jaw assembly 32 is offset slightly to the left, while the elongated shaft 14 extends substantially straight from the pivot assembly 30 to the handle assembly 12.

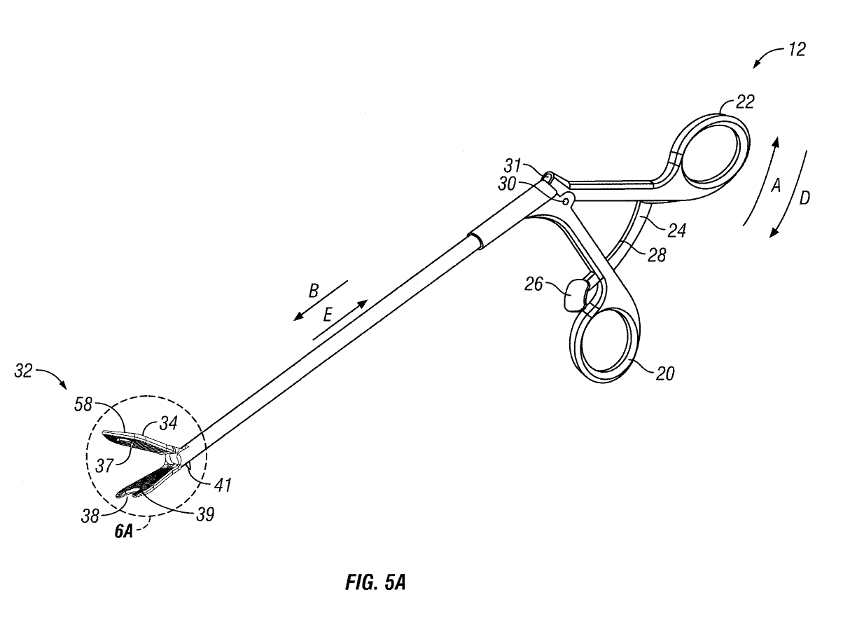

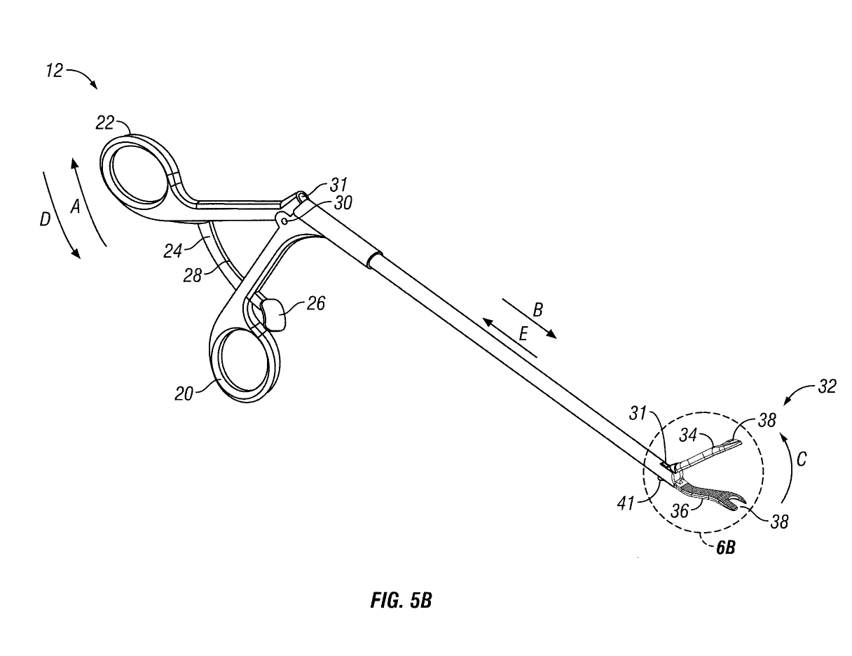

FIGS. 5A and 5B depict perspective views of two illustrative embodiments of a surgical device 10 with the jaw assembly 32 in an open position. As shown in each of the figures, the handle assembly 12 is fully extended, with the thumb hole portion 20 and the finger hole portion 22 fully separated so that the release portion 26 of the ratcheting system 24 abuts against the thumb hole portion 20. The movement of the finger portion 22 in the direction of arrow A causes the transmitting rod 31 to pull back on the jaw assembly 32 in the direction of arrow B, which in turn causes the upper jaw 36 to be pulled away from the lower jaw 34 and in the direction indicated by arrow C.

FIG. 5A provides an illustration of a surgical device 10 configured with a spike 37 protruding from the distal side of the upper jaw 34. The upper jaw spike 37 is positioned to correspond to a spike aperture 39 in the lower jaw 36 so that when the jaw assembly 32 is clamped the upper jaw spike 37 may pass into the spike aperture 39. The spike aperture 39 may either be a through hole in the lower jaw 36 or an indentation. The spike 37 and spike aperture 39 allow for improved grasping of soft tissue by having the spike 37 pierce through the soft tissue. Referring now to FIG. 5B, an alternative embodiment of jaw assembly 32 without an upper jaw spike is provided. In the embodiments of FIGS. 5A and 5B, the underside of distal end 18 of the elongated shaft 14 may include an underside protrusion 41 which may be placed into a tap in the bone for stability. Alternatively, the underside protrusion may be placed on the lower jaw 36.

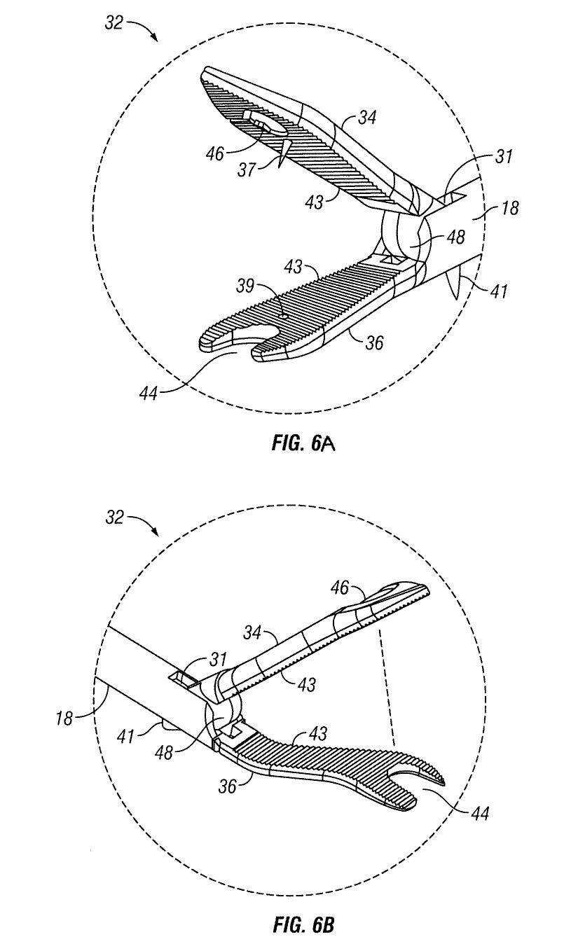

FIGS. 6A and 6B depict a close-up views of the opened jaw assemblies 32 of FIGS. 5A and 5B, respectively. In each of FIGS. 6A and 6B, the jaw assembly 32 includes the upper jaw 34 extended away from lower jaw 36 to reveal a series of grooves or teeth 43 on the inner (or gripping) portion of the upper and lower jaw elements. These grooves 43 provide gripping of soft tissue when the jaw assembly 32 is clamped. The grooves 43 on the underside of upper jaw 34 may be positioned to align with the grooves 43 on the top side of lower jaw 36 so that the indentations of the lower grooves receive the ridges of the upper grooves to form a more secure gripping mechanism. As shown in FIG. 6A (and previously in FIG. 5A), the upper jaw may further include an upper jaw spike 37 and a corresponding spike aperture 39 in a corresponding position on the lower jaw 36 which receives the spike when the jaw assembly 32 is clamped. FIG. 6B provides an alternate configuration without a spike 37. The surgical devices may also include an underside protrusion to stabilize the device. In yet another embodiment, the grooves on the upper or lower jaw may be replaced or supplemented with one or more fang-like teeth, such as spike 37, which may be sufficiently sharp to penetrate soft tissue and provide a stronger grip on the tissue being held. Any suitable tissue gripping features or shapes may be used in the jaw assembly 32.

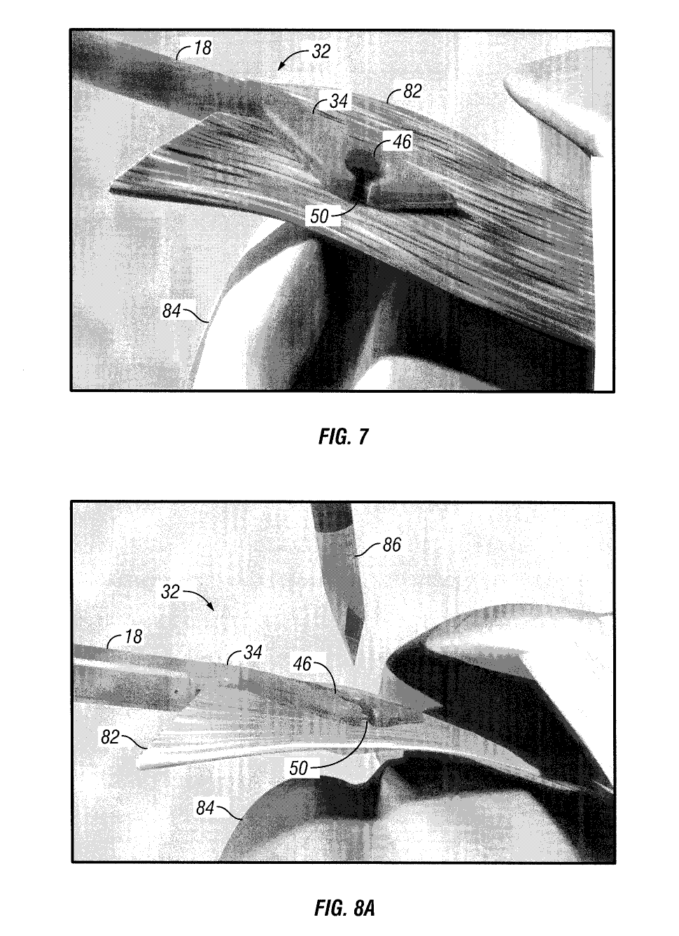

The aperture 38 in the jaw assembly 32 may be made up of two separate apertures—one in the upper jaw 34 and another in the lower jaw 36. In one embodiment, the lower jaw aperture 44 may have a semi-circular shape with one side open such as shown in FIG. 6. A semi-circular lower jaw aperture 44 configuration may reduce the tendency for sutures to be snagged or hung up when removing the grasper from the tissue. In addition, the open configuration may reduce the tendency for the grasper jaws to inadvertently snag tissue when the surgical device releases the tissue and is retracted. In some embodiments, a slot instead of an open side may be included in lower jaw aperture 44. Upper jaw aperture 46 may be generally circular in shape so as to confine any items, such as a suture anchor for example, passing through. In some embodiments, the upper jaw aperture 46 may also include a slot 50 (see FIGS. 7-12, not shown in FIG. 6B) which may allow an item, such as a suture, passing through the upper jaw aperture 46 to be slid out from the aperture, without having to be cut. In some embodiments, the open side of the lower jaw aperture 44 may be positioned at a perpendicular angle to the slot 50 in the upper jaw aperture 46 to further confine a suture anchor within the apertures to prevent it from sliding out accidentally. In these embodiments, the removal of a suture from the upper and lower apertures may include a two step process, first sliding the suture through the upper jaw slot 50 by moving the jaw assembly 22 in a one direction, and then sliding the suture through the lower jaw slot by maneuvering the jaw assembly 22 at a perpendicular angle to the first movement. Also apparent from the close up view of FIGS. 6A and 6B is a jaw pivot assembly 48. The jaw pivot assembly 48 may receive the proximal end of the lower jaw 36 and upper jaw 34. In one embodiment, the jaw pivot assembly 48 may be a pin coupling, with the upper jaw being pivotably moveable along an axis defined by the pin coupling and in the direction indicated by the arrow C. In other embodiments, a pinless assembly may be used. The distal end 18 of the elongated shaft 14 may also include the underside protrusion 41. As noted previously, the underside protrusion may generally take the form of a spike with a sharpened tip that can penetrate soft tissue and bone.

Method of Grasping Soft Tissue and Attaching the Tissue to Bone

In some embodiments, the surgical device 10 may be used to grasp soft tissue in order to hold the soft tissue in place while a bone piercing anchor is pushed through the soft tissue and into the underlying bone. FIGS. 7 through 12 describe one embodiment of using the surgical instrument 10 to more accurately and easily insert a bone-piercing anchor.

FIG. 7 provides an example of how a grasper such as surgical device 10 can be used to grasp soft tissue. As described above, the elongated shaft 14 of surgical device 10 includes a distal end 18. Attached to the distal end 18 of the elongated shaft is the jaw assembly 32 which includes an upper jaw 34 and a lower jaw 36 (not shown). In FIG. 7, the operator of the surgical instrument has caused the jaw assembly 32 to clamp on soft tissue 82, and has pulled the soft tissue away from the underlying bone 84. As is apparent in the drawing, the upper jaw aperture 46 provides an opening to the soft tissue 82. A corresponding opening created by lower jaw aperture 44, (not shown), is positioned beneath the soft tissue 82 in approximately the same location relative to the soft tissue as the upper jaw aperture. In some embodiments, the surgical device may also be used to simultaneously grasp the soft tissue and a biologic tissue augment placed over the soft tissue.

In certain embodiments, the surgical device 10 may be adapted for use in a specific type of surgery. For example, in rotator cuff repair surgery, it may be advantageous to place the anchor suture at a specific distance from the end of the tendon being repaired. In one embodiment, this distance is 15 millimeters. Thus, in one embodiment, the center of the jaw aperture 38 is placed at a predetermined distance, such as exactly 15 millimeters from the proximal edges of the jaws. When the tendon is moved into the open jaw assembly so that it is pushed against the pivoting area of the upper and lower jaws, the aperture is ideally positioned 15 mm from the edge of the tendon. It will be appreciated that other jaw configurations may be used depending on the procedure being performed and the desired suture anchor positions.

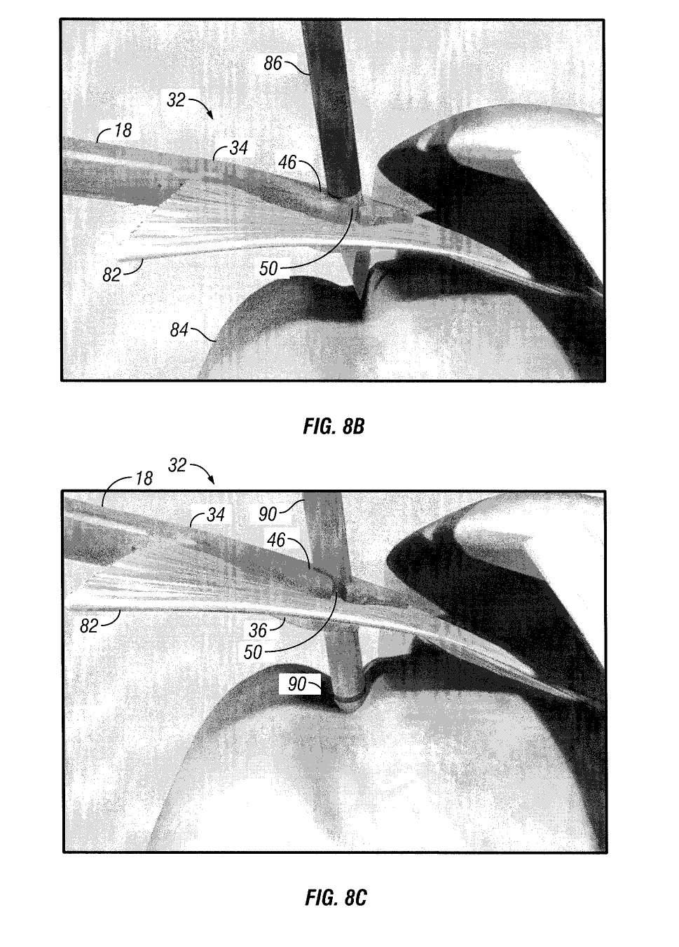

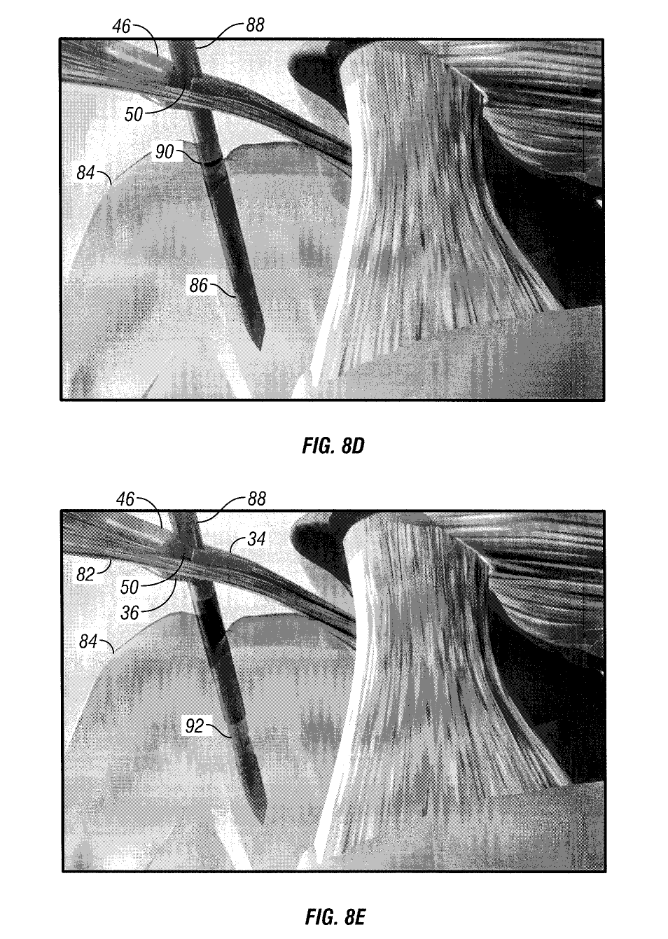

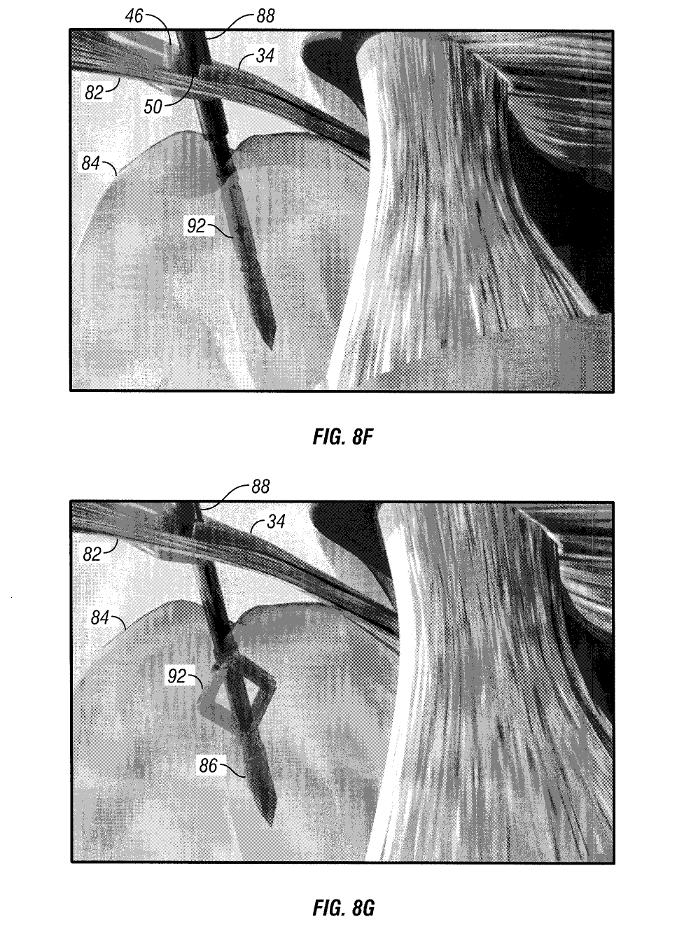

FIGS. 8A-8H illustrate how a suture anchor 86 can be inserted through the soft tissue 82 (or alternatively, through the soft tissue and biologic tissue augment material) and into the bone 84 using the surgical instrument 10 as a guide and stabilizing instrument. Referring now to FIG. 8A, the jaw assembly 32 of the surgical device 10 has been moved into a position above the bone 84 suitable for receiving a suture anchor 86. The suture anchor 86 is of size that fits through the upper jaw aperture 44 and lower jaw aperture 46. In one embodiment, the suture anchor 86 is a bone anchor adapted for piercing through the soft tissue and into underlying bone. The suture anchor 86 preferably has a structure for retaining the anchor in the bone using any suitable retaining technique. Such technique includes, e.g., threading, deployment of retaining structures, and deformation of the anchor. For example, a deformable anchor may include a substantially hollow cylinder with a plurality of cuts in the side of the cylinder. In one embodiment, suture material 96 (now shown) may be pre-attached to the suture anchor 86 so that after implantation, a suture extends from the bone anchor through to the top of the soft tissue for easy passing over the soft tissue (or over both soft tissue and a biologic tissue augment). In one embodiment, the piercing bone anchor has two configurations, a first configuration having a small diameter for easy piercing through soft tissue and bone and a second deployed configuration where structures such as protrusions are deployed to prevent the bone anchor from being easily removed from the bone, such as the anchor described in U.S. patent application Ser. No. 11/143,007, herein incorporated by reference. Although a particular configuration for the suture anchor 86 is described herein, one of skill in the art will appreciate that the suture anchor can be of many different forms including nail type or screw type anchors, so long as it fits within the aperture 38 of the jaw assembly 32.

Once the suture anchor 86 has been moved into place above the upper jaw aperture 46, it is then pushed through the upper aperture 46, the soft tissue 82 (and biologic tissue augment material if present), and lower aperture 44 as shown in FIG. 8B. Once the tip of the suture anchor 86 passes through the aperture 38 of the surgical device 10, the surgical device 10 provides stability to the suture anchor and helps to keep it upright while it is pushed further down through the soft tissue 82 and into bone 84, as shown in FIG. 8B. In addition, the surgical device 10 may be used to hold the soft tissue away from the bone during anchor insertion so that the surgeon can view the anchor as it is being inserted into the bone. The suture anchor 86 may be inserted into the bone 84 by tapping on an inserting device 88 that is in contact with suture anchor, or by any other suitable means of applying axial force to the suture anchor 86.

FIG. 8C shows the suture anchor 86 being inserted further into the soft tissue 82 and bone 84, being pushed by an inserting device 88, until it is fully inserted into the bone 84 as shown in FIG. 8D. The inserting device 88 may include a marker 90 to provide an indication that the suture anchor 86 has been inserted to an appropriate depth. In some embodiments, once the suture anchor 86 is inserted to the appropriate depth in the bone 84, an outer sheath on the inserting device 88 may be retracted from suture anchor as shown in FIG. 8E. As the outer sheath on the inserting device 88 is retracted from the bone 84, a deployment mechanism 92 becomes fully exposed, as illustrated in FIG. 8F.

In one embodiment, the suture anchor deployment mechanism 92 may include a plurality of cuts in the side of the suture anchor. Once the suture anchor 86 has been fully inserted into the bone 84, axial pressure may then be applied to suture anchor 86 to cause it to begin to collapse into a deployed state as shown in FIG. 8G. As the axial pressure continues to be applied (by inserting device 88, or some other device), the suture anchor 86 enters a fully deployed state, as illustrated in FIG. 8H.

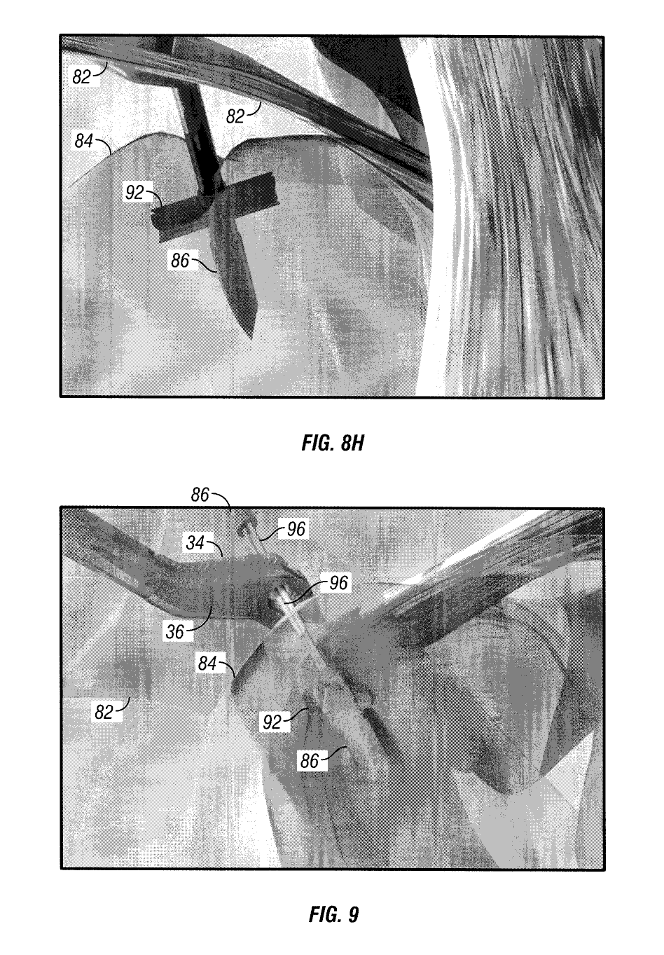

Once the suture anchor is properly inserted and deployed into the bone 84, the inserter device 88 may be detached from the anchor and withdrawn from the bone 84 and from the soft tissue 82 through the aperture 38 of surgical device 10. Removal of the inserter device 88 may expose suture material 96, which has been pre-attached to the suture anchor 86 so that it extends from the anchor 86 through the soft tissue 82 (and biologic tissue augment material if present) and through the aperture 38, as shown in FIG. 9.

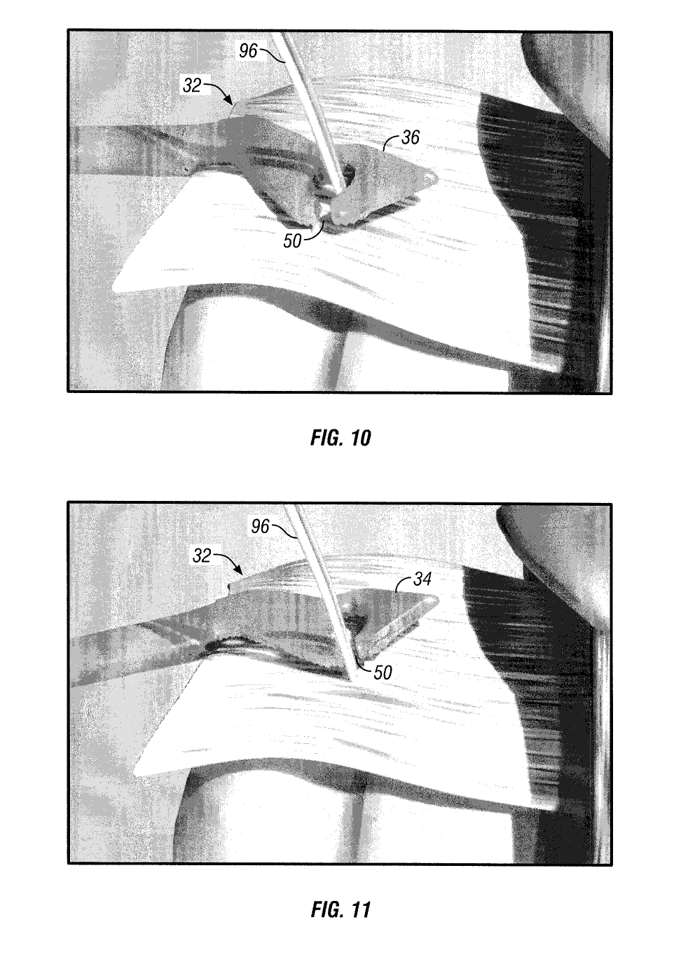

FIG. 10 depicts a top-perspective view of the suture material 96 extending through the soft tissue 92 which is in the grip of the jaw assembly 32 of the surgical device 10. Once the suture material 96 has been anchored to the bone 84, it may no longer be necessary to grasp the soft tissue to hold it in place. As a result, the surgeon may wish to unclamp the jaw assembly 32 and remove the surgical device 10. In order to accomplish the removal of the surgical device 10, the jaw assembly 32 may be maneuvered away from the suture material 96 to allow the suture material 96 to pass through the upper jaw aperture slot 50. Recalling from FIG. 6, that the lower jaw aperture 44 may not be closed, similar maneuvers may not be necessary for removing the lower jaw 36 from its position around the suture material 96. However, as FIGS. 6 and 10 are merely illustrative embodiments, one of skill in the art would readily appreciate that the lower jaw aperture could be configured similarly to the upper jaw aperture, and could also include a slot.

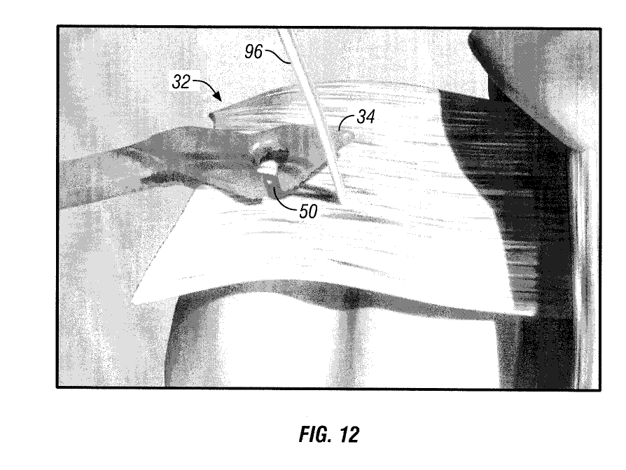

FIG. 11 depicts how the jaw assembly 32 of surgical device 10 can be slidably moved to release the suture material 96 from within the upper jaw aperture 46. As shown in FIG. 11, unclamped jaw assembly 32 is maneuvered so that it may slide the suture material 96 through the upper jaw aperture slot 50. Once the suture material 96 has been positioned beyond the confines of the upper jaw aperture, the jaw assembly may be pulled away from the suture material 96 as shown in FIG. 12. After the surgical device 10 has been removed, the suture material 96 may be passed over the soft tissue and coupled to one or more other anchors such as is described in U.S. patent application Ser. No. 11/143,007, which is incorporated herein by reference in its entirety. When a biologic tissue augment is present, the suture 96 may hold both the soft tissue against bone and the augment against soft tissue.

Although the invention has been described with reference to embodiments and examples, it should be understood that numerous and various modifications can be made without departing from the spirit of the invention. Accordingly, the invention is limited only by the following claims.

What is claimed is:

1. A surgical device for use in grasping tissue, comprising:
a handle assembly;
a jaw assembly; and
a shaft connecting the handle assembly to the jaw assembly,
wherein the jaw assembly includes a first jaw and a second jaw, the first jaw including a first aperture with a first aperture slot configured to allow disengagement of a suture passing through the first aperture, and wherein the second jaw includes a second aperture.

2. The surgical device of claim 1, wherein the first jaw further includes a spike extending in the direction of the second jaw when the jaws are juxtaposed, and the second jaw further includes an aperture positioned to receive the spike.

3. The surgical device of claim 1, further comprising a protrusion positioned on the underside of the shaft.

4. The surgical device of claim 1, further comprising a protrusion positioned on the underside of the second jaw.

5. The surgical device of claim 1, wherein the first jaw has a sharpened tip adapted to facilitate percutaneous insertion of the jaw assembly.

6. The surgical device of claim 5, wherein the sharpened tip is beak-shaped.

7. The surgical device of claim 1, wherein the second aperture includes a second aperture slot which allows disengagement of a suture passing through the second aperture.

8. The surgical device of claim 7, wherein the first and second aperture slots are not aligned.

9. The surgical device of claim 1, wherein the first jaw and the second jaw are coupled through a jaw pivot positioned at a proximal end of the first jaw and a proximal end of the second jaw.

10. A method of attaching soft tissue to bone, comprising:
grasping the soft tissue with jaws of a surgical device, the jaws of the surgical instrument including an aperture;
passing a suture anchor through the aperture, the suture anchor having a pre-attached suture; and
inserting the suture anchor into the bone.

11. The method of claim 10, further comprising placing a tissue augment over the soft tissue prior to grasping the soft tissue, then grasping both the tissue augment and the soft tissue with the jaws of the surgical device.

12. The method of claim 1 1, further comprising passing the suture over the tissue augment and the soft tissue and attaching the suture to a second suture anchor.

13. The method of claim 10, further comprising, prior to inserting the suture anchor into the bone, placing a protrusion on the surgical instrument into an indentation formed in the bone.

14. A tissue grasper configured to grasp soft tissue in vivo comprising:
an upper jaw comprising a substantially circular first aperture and a first opening on a side of the upper jaw leading into the first aperture; and
a lower jaw comprising a substantially circular second aperture and a second opening on a side of the lower jaw leading into the second aperture.

15. The grasper of claim 14, wherein the first and second openings are configured to allow a suture that is passing through the first and second apertures to be disengaged from the tissue grasper by moving the suture laterally through the first and second openings.

16. The grasper of claim 14, wherein the first opening comprises a slot on the side of the upper jaw.

17. The grasper of claim 14, wherein the second opening comprises a slot on the side of the lower jaw.

18. The grasper of claim 14, wherein the first opening is not aligned with the second opening.

19. The grasper of claim 14, wherein the upper jaw comprises a tissue contacting surface and at least a portion of interior walls of the first aperture are at a non-perpendicular angle relative to the tissue contacting surface.

20. The grasper of claim 14, wherein the lower jaw comprises a tissue contacting surface and at least a portion of interior walls of the second aperture are at a non-perpendicular angle relative to the tissue contacting surface.
